(19) United States
(12) Patent Application Publication
(10) Pub. No.: US 2005/0037390 A1
(43) Pub. Date: Feb. 17, 2005

(54) Methods and compositions for modulating erythropoietin expression

(75) Inventors: Fred King, Encinitas, CA (US); Sumit Chanda, La Jolla, CA (US); Jeremy S. Caldwell, Cardiff, CA (US); John Hogenesch, Encinitas, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, a Delaware Limited Liability Company, Hamilton (BM)

(21) Appl. No.: 10/862,013

(22) Filed: Jun. 4, 2004

Related U.S. Application Data
(60) Provisional application No. 60/475,605, filed on Jun. 4, 2003.

(51) Int. Cl. C12Q 1/68; G01N 33/53; G01N 33/567; C12N 15/85

(52) U.S. Cl. 435/6; 435/7.2; 435/455

(57) ABSTRACT

This invention provides novel Epo-modulating polypeptides. The invention also provides methods for screening modulators of Epo expression. The methods comprise first screening test agents for modulators of an Epo-modulating polypeptide and then further screening the identified modulating agents for modulators of Epo expression. The invention further provides methods and pharmaceutical compositions for modulating Epo expression in cells and for treating diseases and conditions due to Epo deficiency.

### CROSS-REFERENCE TO RELATED APPLICATIONS

This application claims the benefit of priority to U.S. Provisional Patent Application Ser. No. 60/475,605 (filed Jun. 4, 2003). The full disclosure of the priority application is incorporated herein by reference in its entirety and for all purposes.

### FIELD OF THE INVENTION

The present invention generally relates to methods for identifying modulators of erythropoietin expression and therapeutic applications of such modulators. More particularly, the invention pertains to novel modulators that regulate the expression level of erythropoietin, and to methods of using such modulators to treat diseases or conditions due to erythropoietin deficiency in a subject.

### BACKGROUND OF THE INVENTION

Erythropoietin (Epo) is a glycoprotein hormone that regulates the growth and differentiation of red blood cell (erythrocyte) progenitors. Produced mainly in the fetal liver and adult kidney, Epo induces proliferation and differentiation of red blood cell progenitors through interaction with receptors on the surface of erythroid precursor cells. Erythropoietin enhances erythropoiesis by stimulating formation and proliferation of proerythroblasts into reticulocytes and subsequent release of reticulocytes from bone marrow. Ultimately, erythropoietin stimulates the maturation of reticulocytes into morphologically identifiable red blood cells. Epo also plays a role in other important physiologic functions including mitogenesis, modulation of calcium influx into smooth muscle cells and neural cells, and effects on intermediary metabolism.

During fetal development the liver serves as the primary source of Epo. Shortly before birth, production of Epo in the liver decreases and the kidney becomes the primary source. In both tissues, Epo transcription is subject to physiological regulation at the level of gene transcription in response to hypoxia. Its production, therefore, is principally regulated by the level of renal oxygenation. Approximately 10-15% of erythropoietin is produced by extrarenal sites, including the liver, which seems to be responsible for residual erythropoietin production in anephric patients and in the fetus.

There is a need in the art for novel methods and compositions that are useful in modulating (e.g., enhancing) Epo expression. Such modulation will have significant utility for effective treatment of many diseases and disorders, e.g., those related to Epo deficiency. The instant invention fulfills this and other needs.

### SUMMARY OF THE INVENTION

In one aspect, the present invention provides methods for identifying agents that modulate expression of erythropoietin (Epo). The methods comprise (a) assaying a biological activity of an Epo-modulating polypeptide identified in the invention, or a fragment of said polypeptide, in the presence of a test agent to identify one or more modulating agents that modulate the biological activity of the polypeptide; and (b) testing one or more of the modulating agents for ability to modulate expression of Epo.

In some of these methods, (b) comprises testing the modulating agents for ability to modulate expression of a reporter gene under the control of an Epo transcription regulatory element. The Epo transcription regulatory element can be human Epo 3′ enhancer element.

In some of the methods, the Epo-modulating polypeptide is a transcription regulator and the biological activity is transcriptional regulation. In some methods, the Epo-modulating polypeptide is an enzyme and the biological activity is an enzymatic activity of the Epo-modulating polypeptide. In some methods, the test agent modulates cellular level of the Epo-modulating polypeptide. In some methods, the modulating agent identified in (b) enhances Epo expression.

In some of the methods, the assaying of the biological activity of the Epo-modulating polypeptide occurs in a cell. In some of these methods, the Epo-modulating polypeptide is expressed from said polynucleotide that has been introduced into the cell.

In some methods, the testing for ability to modulate Epo expression comprises (a) providing a cell or cell lysate that comprises a reporter gene that is operably linked to an Epo transcription regulatory element; (b) contacting the cell or cell lysate with a modulating agent; and (c) detecting an increase or decrease in expression of the reporter gene in the presence of the modulating agent compared to expression of the reporter gene in the absence of the modulating agent. In some of the methods, the testing for ability to modulate expression of Epo comprises contacting a cell or cell lysate with the modulating agent and determining cellular level of Epo or a fragment of Epo.

In another aspect of the invention, methods are provided for modulating Epo expression in a cell. These methods comprise contacting the cell with an effective amount of (a) an Epo-modulating polypeptide encoded by a polynucleotide selected from the members listed in Table 1, or a fragment of the Epo-modulating polypeptide, or (b) an Epo modulator identified in accordance with methods of the invention. In some of these methods, expression of human Epo is modulated. In some methods, the cell is a kidney cell or a liver cell. In some methods, the cell is present in a subject. The subject can be human or other non-human mammals.

In some of these methods, the Epo-modulating polypeptide or its fragment is expressed from an expression vector that has been introduced into the cell. In some methods, the Epo modulator employed enhances Epo expression. The Epo modulator can be a small molecule compound.

In another aspect, the invention provides methods for treating erythropoietin deficiency in a subject. Such methods comprise administering to the subject a pharmaceutical composition comprising an effective amount of an Epo-modulator that enhances Epo expression. The Epo-modulator employed in these methods is identified by (a) assaying a biological activity of an Epo-modulating polypeptide identified in the invention, or a fragment of the polypeptide, in the presence of a test agent to identify one or more modulating agents that modulate the biological activity of the polypeptide; and (b) testing one or more of the modulating agents for ability to enhance expression of Epo gene.

In some of these methods, the subject is a human. Preferably, the Epo modulator employed in these methods is a small molecule compound. In some of these methods, the Epo-modulating polypeptide is a transcription regulator. In some methods, the Epo-modulating polypeptide is an enzyme. Some of the methods are directed to treating subjects suffering from anemia.

A further understanding of the nature and advantages of the present invention may be realized by reference to the remaining portions of the specification and claims.

### DETAILED DESCRIPTION

The present invention is predicated in part on the discovery that expression of a reporter gene driven by an Epo transcription regulatory element is up-regulated by a number of polypeptides of diverse biochemical properties. As shown in Table 1 and described in more detail in the Examples below, these Epo-modulating polypeptides enhance expression of the reporter polypeptide under the control of the 3′ enhancer of human Epo gene. In addition, it was discovered by the present inventors that some of these Epo-modulating polypeptides modulate expression mediated by the Epo 3′ enhancer by stabilizing HIF1-α (see Example 2). HIF1-α is a component of a protein complex that binds to the Epo 3′ element and activates downstream gene expression.

In accordance with these discoveries, the present invention provides novel modulators of Epo expression and methods for identifying such modulators. The invention also provides methods for modulating Epo expression in a cell and for treating diseases or conditions due to abnormal Epo level or Epo expression in a subject. The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., polypeptides or small molecule compounds) or combining agents and cells (e.g., a polypeptide and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein and are generally available.

A “host cell,” as used herein, refers to a prokaryotic or eukaryotic cell into which a heterologous DNA has been or will be introduced. The heterologous DNA can be introduced into the cell by any means, e.g., electroporation, calcium phosphate precipitation, microinjection, transformation, viral infection, and/or the like.

The terms “identical”, “sequence identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, e.g., an Epo-modulating polypeptide encoded by a polynucleotide in Table 1, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., a polynucleotide in Table 1, e.g., as measured by BLASTN (or CLUSTAL, or any other available alignment software) using default parameters.

The term “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “isolated” means that the material is removed from its original environment (e.g., the natural environment if it is naturally occurring). For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

The terms “nucleic acid,” “DNA sequence,” and “polynucleotide” refer to a deoxyribonucleotide or ribonucleotide polymer in either single- or double-stranded form, and unless otherwise limited, encompass known analogues of natural nucleotides that hybridize to nucleic acids in a manner similar to naturally occurring nucleotides. A “polynucleotide sequence” is a nucleic acid comprised of a polymer of nucleotides (A, C, T, U, G, etc., or naturally occurring or artificial nucleotide analogues) or a character string representing a nucleic acid, depending on the context. Either the given nucleic acid or the complementary nucleic acid can be determined from any specified polynucleotide sequence.

The term “modulate” refers to a change in the cellular level or other biological activities of a reference molecule. Modulation can be up-regulation (i.e., activation or stimulation) or down-regulation (i.e. inhibition or suppression). With respect to modulation of erythropoietin expression level, the change can arise from, for example, an increase or decrease in expression of the Epo gene, stability of mRNA that encodes the Epo protein, translation efficiency, or a change in post-translational modifications or stability of the protein. The mode of action can be direct, e.g., through binding to the reference protein or to genes encoding the reference protein. The change can also be indirect, e.g., through binding to, stabilizing, and/or modifying (e.g., enzymatically) another molecule which otherwise modulates the reference protein.

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The term “operably linked” refers to a functional relationship between two or more polynucleotide (e.g., DNA) segments. Typically, it refers to the functional relationship of a transcriptional regulatory sequence to a transcribed sequence. For example, an Epo promoter or enhancer sequence, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “polypeptide” is used interchangeably herein with the terms “polypeptides” and “protein(s)”, and refers to a polymer of amino acid residues, e.g., as typically found in proteins in nature. A “mature protein” is a protein which is full-length and which, optionally, includes glycosylation or other modifications typical for the protein in a given cell membrane.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or an oncopromoter.

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression cassette.

The term “subject” refers to human and non-human animals. It encompasses human and other mammals. Examples of non-human subjects include cows, horses, sheep, pigs, cats, dogs, mice, rats, rabbits, guinea pigs, monkeys.

Transcription refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilizing the transcript.

A transcription regulatory element or sequence include, but is not limited to, a promoter sequence (e.g., the TATA box), an enhancer element, a signal sequence, or an array of transcription factor binding sites. It controls or regulates transcription of a gene operably linked to it.

A “variant” of a molecule such as an Epo-modulating polypeptide of the invention is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A “vector” is a composition for facilitating introduction, replication and/or expression of a selected nucleic acid in a cell. Vectors include, e.g., plasmids, cosmids, viruses, YACs, bacteria, poly-lysine, etc. A “vector nucleic acid” is a nucleic acid molecule into which heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

II. Identification of Novel Epo-Modulating Polypeptides

Human Epo gene encodes a 30.4 kD glycoprotein. The structure and functions of erythropoietin have been characterized and described in the art, e.g., Goldberg et al., Biology of erythropoietin, p. 59-104, in Erythropoietin in clinical applications: an international perspective, Garnick, M. B. (ed.), Marcel Dekker, Inc., New York, 1990; Jelkmann, Physiol. Rev. 72: 449-89, 1992; and Krantiz, Blood 77: 419-34, 1991. Epo genes from human and various other species have been cloned and characterized. See, e.g., U.S. Pat. No. 4,703,008; Blanchard et al., Mol. Cell. Biol. 12: 5373-85, 1992; Nagao et al., Biochim Biophys Acta. 1171:99-102, 1992; and Pugh et al., Biochim Biophys Acta. 1217: 297-306, 1994.

The present invention provides novel protein or polypeptide modulators that modulate erythropoietin expression. Utilizing an expression vector which expresses a reporter gene under the control of an Epo enhancer element, a number of polynucleotides were identified which up-regulate expression of the reporter gene when the expression vector and the polynucleotides were co-transfected into a host cell (see Examples below). Table 1 lists exemplary polynucleotides encoding such Epo-modulating polypeptides. As shown in the Table, the novel Epo-modulating polypeptides include very diversified classes of proteins, including DNA-binding protein (e.g., Clone No. 18), other transcription regulatory proteins (e.g., Clone No. 21), RNA binding proteins (e.g., Clone No. 32), proteases (e.g., Clone No: 39), and other enzymes (e.g., Clone Nos. 23 and 35).

Other than directly modulating expression (e.g., transcription or translation) of the Epo gene, the Epo-modulating polypeptides can also modulate other transcription factors or proteins that are involved in Epo expression. A number of proteins are known to be involved in regulation of Epo expression. For example, human Epo expression is negatively regulated by the transcription factors hGATA-1, 2, or 3 (Imagawa et al., Blood 89: 1430-9, 1997). Up-regulation of Epo gene transcription in hypoxia depends on at least two known DNA binding transcription factors, HIF-1 and HNF-4. These factors bind to cognate response elements in a 3′ enhancer of the Epo gene (Huang et al., Kidney Int 51: 548-52, 1997). Further, post-transcriptional regulation of erythropoietin mRNA stability was also observed. It was shown that erythropoietin mRNA-binding protein (ERBP) binds to the 3′-untranslated region (3′-UTR) of erythropoietin (Epo) mRNA and enhances the stability of the Epo mRNA (McGary et al., J Biol Chem 272: 8628-34, 1997). As demonstrated in Example 2 below, the Epo-modulating polypeptides of the invention can indirectly modulate Epo expression by modulate activities, stability, or cellular levels of any of these proteins or factors (e.g., HIF1-α) that directly modulate Epo gene or Epo mRNA.

III. Methods for Screening Modulators of Epo Expression

A. General Scheme and Assay Systems

The Epo-modulating polypeptides described above provide novel targets for screening modulators (stimulators or inhibitors) of Epo expression. Employing these Epo-modulating polypeptides, the present invention provides methods for screening agents or compounds that modulate Epo expression. Various biochemical and molecular biology techniques well known in the art can be employed to practice the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999).

In some methods, test agents are first assayed for their ability to modulate a biological activity of an Epo-modulating polypeptide (“the first assay step”). Modulating agents thus identified are then subject to further screening for ability to modulate Epo expression, typically in the presence of the Epo-modulating polypeptide (“the second testing step”). Depending on the Epo-modulating polypeptide employed in the method, modulation of different biological activities of the Epo-modulating polypeptide can be assayed in the first step. For example, a test agent can be assayed for binding to the Epo-modulating polypeptide. The test agent can be assayed for activity to modulate expression level of the Epo-modulating polypeptide, e.g., transcription or translation. The test agent can also be assayed for activities in modulating cellular level or stability of the Epo-modulating polypeptide, e.g., post-translational modification or proteolysis. If the Epo-modulating polypeptide has a known or well established biological function (e.g., enzymatic activity, or DNA-binding activity), the biological activity monitored in the first assay step can be the specific biochemical or enzymatic activity of the Epo-modulating polypeptide.

Once test agents that modulate the Epo-modulating polypeptides are identified, they are typically further tested for ability to modulate Epo expression. If a test agent identified in the first assay step modulates cellular level (e.g., by altering transcription activity) of the Epo-modulating polypeptide, it would indirectly modulate Epo expression. On the other hand, if a test agent modulates an activity other than cellular level of the Epo-modulating polypeptide, then the further testing step is needed to confirm that their modulatory effect on the Epo-modulating polypeptide will indeed lead to modulation of Epo expression. For example, a test agent that modulates DNA-binding activity of an Epo-modulating polypeptide needs to be further tested in order to confirm that modulation of the DNA-binding activity of the Epo-modulating polypeptide can result in modulation of Epo expression.

Both stimulators and inhibitors of Epo expression can be identified with methods of the invention. As noted above, all the Epo-modulating polypeptides in Table 1 enhance Epo expression. Therefore, modulating agents identified in the first assay step that stimulate expression or biological activities of the Epo-modulating polypeptide are likely to also enhance Epo expression. Conversely, modulating agents identified in the first screening step that suppress expression or biological activities of the Epo-modulating polypeptide are more likely to be inhibitors of Epo expression. Nevertheless, the exact effects on Epo expression by the modulating agents identified in the first screening step can only be determined in the second testing step.

A variety of routinely practiced assays can be employed in the first assay step and the second testing step. Cell-based screening systems can be used to screen test agents that modulate the Epo-modulating polypeptide or further examine modulating agents thus identified for ability to alter Epo expression. For example, to identify test agents that modulate expression of the Epo-modulating polypeptide in the first assay step, a construct comprising a transcription regulatory element of the polypeptide that is operably linked to a reporter gene can be introduced into a host cell system. Test agents are then examined for ability to alter the reporter gene expression. The reporter gene activity (e.g., an enzymatic activity) in the presence of a test agent can be determined and compared to the activity of the reporter gene in the absence of the agent. An increase or decrease in the activity identifies a modulator of expression of the Epo-modulating polypeptide. The reporter gene used in such systems can encode any detectable polypeptide (response or reporter polypeptide) known in the art, e.g., detectable by fluorescence or phosphorescence or by virtue of its possession of an enzymatic activity. The detectable response polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

In addition to the first assay step, the cell-based systems can also be employed in the second testing step to examine modulating agents identified in the first step for ability to modulate Epo expression, as detailed below. Further, other than monitoring expression of a reporter gene, the cell-based systems can also measure activities of Epo or the Epo-modulating polypeptide. For example, in the first assay step, the assay systems can be designed to directly monitor cellular level or a biological activity (e.g., enzymatic activity) of the Epo-modulating polypeptide. In the second testing step, the assay system can monitor expression of Epo gene itself in the presence or absence of the modulating agents identified in the first assay step.

In the cell-based assays, the test agents or the modulating agents (e.g., peptides or polypeptides) can also be expressed from a different vector that is also present in the host cell. In some methods, a library of test agents are encoded by a library of such vectors (e.g., a cDNA library as employed in the Examples below). Such libraries can be generated using methods well known in the art (see, e.g., Sambrook et al. and Ausubel et al., supra) or obtained from a variety of commercial sources.

In addition to cell based assays described above, modulators of Epo expression can also be screened with non-cell based methods. These assays can be employed to identify agents that bind to an Epo-modulating polypeptide or modulate expression of the Epo-modulating polypeptide (e.g., in the first screening step). Such methods include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel et al., supra (chapter 12, DNA-Protein Interactions). One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin (1996) Am. J. Hum. Genet. 59:561-569; Tang (1996) Biochemistry 35:8216-8225; Lingner (1996) Proc. Natl. Acad. Sci. USA 93:10712; Chodosh (1986) Mol. Cell. Biol 6:4723-4733.

In both the first assay step and the second testing step, an intact Epo-modulating polypeptide or its fragments, analogs, or functional derivatives can be used. The fragments that can be employed in these assays usually retain one or more of the biological activities of the Epo-modulating polypeptide, e.g., DNA-binding activity if the Epo-modulating employed in the first assay step is a DNA-binding protein. Fusion proteins containing such fragments or analogs can also be used for the screening of test agents. Functional derivatives of Epo-modulating polypeptides usually have amino acid deletions and/or insertions and/or substitutions while maintaining one or more of the bioactivities and therefore can also be used in practicing the screening methods of the present invention. A functional derivative of an Epo-modulating polypeptide can be prepared from a naturally occurring or recombinantly expressed protein by proteolytic cleavage followed by conventional purification procedures known to those skilled in the art. Alternatively, the functional derivative can be produced by recombinant DNA technology by expressing only fragments of an Epo-modulating polypeptide that retains one or more of their bioactivities.

B. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000 or 500). Preferably, high throughput assays are adapted and used to screen for such small molecules. In some methods, combinatorial libraries of small molecule compounds as described above can be readily employed to screen for small molecule modulators of Epo expression. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1 :384-91.

Libraries of test agents to be screened with the claimed methods can also be generated based on structural studies of the Epo-modulating polypeptides, their fragments or analogs. Such structural studies allow the identification of test agents that are more likely to bind to the Epo-modulating polypeptides. The three-dimensional structure of an Epo-modulating polypeptide can be studied in a number of ways, e.g., crystal structure and molecular modeling. Methods of studying protein structures using x-ray crystallography are well known in the literature. See Physical Biochemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of Epo-modulating polypeptides' structures provides another means for designing test agents for screening Epo modulators. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to an Epo-modulating polypeptide in Table 1. Such antibodies can be monoclonal or polyclonal. Such antibodies can be generated using methods well known in the art. For example, the production of non-human monoclonal antibodies, e.g., murine or rat, can be accomplished by, for example, immunizing the animal with an Epo-modulating polypeptide or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to an Epo-modulating polypeptide of the present invention.

Human antibodies against an Epo-modulating polypeptide can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an Epo-modulating polypeptide or its fragment.

C. Screening Test Agents that Modulate Epo-Modulating Polypeptides

A number of assay systems can be employed to screen test agents for modulators of an Epo-modulating polypeptide. As noted above, the screening can utilize an in vitro assay system or a cell-based assay system. In this screening step, test agents can be screened for binding to the Epo-modulating polypeptide, altering cellular level of the Epo-modulating polypeptide, or modulating other biological activities of the Epo-modulating polypeptide.

1. Binding of Test Agents to an Epo-Modulating polypeptide

In some methods, binding of a test agent to an Epo-modulating polypeptide is determined in the first assay step. Binding of test agents to an Epo-modulating polypeptide can be assayed by a number of methods including, e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Eckeret al., Bio/Technology 13:351-360, 1995; and Hodgson, Bio/Technology 10:973-980, 1992. The test agent can be identified by detecting a direct binding to the Epo-modulating polypeptide, e.g., co-immunoprecipitation with the Epo-modulating polypeptide by an antibody directed to the Epo-modulating polypeptide. The test agent can also be identified by detecting a signal that indicates that the agent binds to the Epo-modulating polypeptide, e.g., fluorescence quenching.

Competition assays provide a suitable format for identifying test agents that specifically bind to an Epo-modulating polypeptide. In such formats, test agents are screened in competition with a compound already known to bind to the Epo-modulating polypeptide. The known binding compound can be a synthetic compound. It can also be an antibody, which specifically recognizes the Epo-modulating polypeptide, e.g., a monoclonal antibody directed against the Epo-modulating polypeptide. If the test agent inhibits binding of the compound known to bind the Epo-modulating polypeptide, then the test agent also binds the Epo-modulating polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an Epo-modulating polypeptide or its fragments onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the Epo-modulating polypeptide, the test agents are bound to the solid matrix and the Epo-modulating polypeptide molecule is then added.

Soluble assays include some of the combinatory libraries screening methods described herein. Under the soluble assay formats, neither the test agents nor the Epo-modulating polypeptide are bound to a solid support. Binding of an Epo-modulating polypeptide or fragment thereof to a test agent can be determined by, e.g., changes in fluorescence of either the Epo-modulating polypeptide or the test agents, or both. Fluorescence may be intrinsic or conferred by labeling either component with a fluorophor.

In some binding assays, either the Epo-modulating polypeptide, the test agent, or a third molecule (e.g., an antibody against the Epo-modulating polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

2. Agents Modulating Other Activities of Epo-Modulating Polypeptides

Binding of a test agent to an Epo-modulating polypeptide provides an indication that the agent can be a modulator of the Epo-modulating polypeptide. It also suggests that the agent may modulate Epo expression (e.g., by binding to and modulate the Epo-modulating polypeptide which in turn acts on Epo expression). Thus, a test agent that binds to an Epo-modulating polypeptide can be further tested for ability to modulate Epo expression (i.e., in the second testing step outlined above). Alternatively, a test agent that binds to an Epo-modulating polypeptide can be further examined to determine its activity on the Epo-modulating polypeptide. The existence, nature, and extend of such activity can be tested by an activity assay. Such an activity assay can confirm that the test agent binding to the Epo-modulating polypeptide indeed has a modulatory activity on the Epo-modulating polypeptide.

More often, an activity assays can be used independently to identify test agents that modulate activities of an Epo-modulating polypeptide (i.e., without first assaying their ability to bind to the Epo-modulating polypeptide). These include assaying effects on expression or cellular level of the Epo-modulating polypeptide or assaying effects on other biological activities of the Epo-modulating polypeptide (e.g., a DNA-binding activity or an enzymatic activity). In general, such methods involve adding a test agent to a sample containing an Epo-modulating polypeptide in the presence or absence of other molecules or reagents which are necessary to test a biological activity of the Epo-modulating polypeptide (e.g., DNA-binding activity if the Epo-modulating polypeptide is a DNA-binding protein), and determining an alteration in the biological activity of the Epo-modulating polypeptide. In addition to assays for screening agents that modulate an enzymatic or other biological activities of an Epo-modulating polypeptide, the activity assays also encompass in vitro screening and in vivo screening for alterations in expression or cellular level of the Epo-modulating polypeptide.

In an exemplary embodiment, the Epo-modulating polypeptide is a DNA-binding protein, e.g., E12 protein (Clone No. 21 in Table 1), the test agent can be first examined for its activity in modulating DNA-binding of the Epo-modulating polypeptide. Methods to monitor DNA-binding activity of E12 have been described in the art, e.g., in Rashbass et al., EMBO J. 11: 2981-90, 1992; and Sawada et al., Mol Cell Biol 13: 5620-8, 1993. Similarly, if the Epo-modulating polypeptide employed in the first screening step is an enzyme (e.g., methylenetetrahydrofolate reductase encoded by Clone No. 23 in Table 1), the test agent is examined for ability to modulate the enzymatic activity of the Epo-modulating polypeptide. Methods for monitoring various enzyme activities have been described in the art. For example, methylenetetrahydrofolate reductase catalyses the reduction of methylenetetrahydrofolate to methyltetrahydrofolate, and its activity can be assayed as described in, e.g., Frosst et al., Nat Genet 10: 111-3, 1995; and Goyette et al., Nat Genet 7: 195-200, 1994.

D. Screening Agents that Modulate Epo Expression

Once a modulating agent has been identified to bind to an Epo-modulating polypeptide and/or to modulate a biological activity (including cellular level) of the Epo-modulating polypeptide, it can be further tested for ability to modulate Epo expression. Modulation of Epo expression by the modulating agent is typically tested in the presence of the Epo-modulating polypeptide. When a cell-based screening system is employed, the Epo-modulating polypeptide can be expressed either endogenously or from an expression vector that has been introduced into a host cell.

As noted above, various assay formats can be employed in this screening step. Similar to the first screening step for identifying agents that modulate Epo-modulating polypeptides, modulation of Epo expression can be determined in a non-cell based assay system or cell-based assays. Using eukaryotic in vitro transcription systems, effects of modulating agents on Epo expression can be tested by directly measuring Epo transcription activity in the presence of the agents. Such in vitro assays can be performed using routinely practiced methods described in the art, e.g., Sambrook et al., supra; and Ausubel et al., supra. Because the test agent is likely to exert its modulatory effect on the Epo by modulating an Epo-modulating polypeptide, the Epo-modulating polypeptide is typically also present in such non-cell based assay systems.

Preferably, the modulating agents identified in the first screening step are further examined with a cell based assay system. Similar to the first screening step, modulation of Epo expression can be examined in a cell-based system by transient or stable transfection of an expression vector into cultured cell lines. The expression vector can contain an Epo transcription regulatory element operably linked to a reporter gene, and can be prepared using only routinely practiced techniques and methods of molecular biology (see, e.g., Sambrook et al. and Ausubel et al., supra).

In some preferred embodiments, the expression vectors bear the 3′ enhancer element of the human Epo gene and an appropriate promoter that are operably linked to a reporter gene (as described in the Examples below). The 3′ enhancer element of the Epo gene has been described in the art, e.g., Blanchard et al., Mol. Cell. Biol. 12: 5373-85, 1992. Other than the human Epo 3′ enhancer element, sequences substantially identical to the enhancer or the 3′ enhancer element of an Epo gene from other species (e.g., mouse Epo gene; Pugh et al., Biochim Biophys Acta. 1217: 297-306, 1994) can also be used to construct the expression vectors. With the cell-based assays, vectors expressing a reporter gene or other linked polynucleotides under the control of a transcription regulatory element of the Epo gene are introduced into appropriate host cells (e.g., HepG2 cells as described in the Examples). When inserted into the appropriate host cell, the transcription regulatory elements in the expression vector induces transcription of the reporter gene by host RNA polymerases. Reporter genes typically encode polypeptides with an easily assayable enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP).

Modulation of Epo expression is typically examined by measuring expression of the reporter genes or other linked polynucleotides. An agent that modulates an Epo-modulating polypeptide identified in the first screening step is tested for ability to alter the reporter gene expression in the presence of the Epo-modulating polypeptide. Alternatively, instead of a reporter gene, the expression vectors can also harbor the Epo gene under the control of necessary transcription regulatory sequences (e.g., the Epo 3′ enhancer and an appropriate promoter). In these embodiments, the cell based assays directly measure expression of Epo gene in the presence of an Epo-modulating polypeptide and an agent that modulates the Epo-modulating polypeptide.

Expression of Epo in the cell-based systems can be monitored using methods well known in the art (e.g., Sambrook et al., supra; and Ausubel et al., supra) as well as numerous methods described in the literatures, e.g., Maxwell et al., Blood 15: 1823-30, 1994; Chin et al., Nucleic Acids Res 11: 3041-9, 1995; Imagawa, Acta Haematol 95: 248-56, 1996; and La Ferla et al., FASEB J 16: 1811-3, 2002. General methods of cell culture, transfection, and reporter gene assay have been described in the art, e.g., Ausubel, supra; and Transfection Guide, Promega Corporation, Madison, Wis. (1998). Any readily transfectable mammalian cell line may be employed to practice methods of the invention, e.g., HepG2 cell (see Example below); Hep3B cell (Blanchard et al., Mol. Cell. Biol. 12: 5373-85, 1992); COS (e.g., ATCC No. CRL 1650 or 1651); BHK (e.g., ATCC No. CRL 6281); CHO-KI (ATCC No. CCL 61), HeLa (e.g., ATCC No. CCL 2), HEK 293 (ATCC No. 1573) and NS-1 cells.

Transcription driven by an Epo transcription regulatory element may also be detected by directly measuring the amount of RNA transcribed from the reporter gene. In these embodiments, the reporter gene may be any transcribable nucleic acid of known sequence that is not otherwise expressed by the host cell. RNA expressed from constructs driven by the Epo transcription regulatory element may be analyzed by techniques known in the art, e.g., reverse transcription and amplification of mRNA, isolation of total RNA or poly A+ RNA, northern blotting, dot blotting, in situ hybridization, RNase protection, primer extension, high density polynucleotide array technology and the like. These techniques are all well known and routinely practiced in the art.

IV. Therapeutic Applications

Epo production is regulated through hypoxic activation of gene transcription and possibly hypoxia-induced stabilization of its mRNA. In addition to liver and kidney, Epo is also produced in central nervous system and reproductive organs. Epo plays important roles in erythropoiesis. Epo also protects neurons from a various types of damage (Digicaylioglu et al., Nature. 412: 601-2, 2001). The uterine Epo may be involved in the estrogen-dependent angiogenesis of the endometrial layer. Epo could also function in other tissues (see, e.g., Sasaki, Intern Med 42: 142-9, 2003).

As a consequence of the connection between Epo and erythropoiesis and the other physiological activities, modulating expression or cellular level of Epo can lead to modulation of the various cellular processes relating to erythropoiesis as well as various other cellular and physiological activities. Accordingly, the present invention provides compositions and methods for modulating expression of Epo gene in a cell, and for modulating cellular processes mediated by Epo. The invention further provides therapeutic compositions and methods for preventing or treating diseases and conditions due to abnormal expression or cellular level of Epo.

To modulate Epo expression in vivo, a cell (e.g., a kidney cell or a liver cell) can be contacted with any a number of the Epo modulators identified in accordance with the present invention. In some methods, a polynucleotide encoding a modulator of Epo of the present invention is introduced by retroviral or other means. In some methods, a modulator of Epo of the present invention is introduced directly to a subject (e.g., a human or a non-human mammal). For example, a small molecule Epo-modulator that enhances human Epo expression can be administered to a human subject to treat various diseases in which Epo deficiency plays a role.

A. Disease and Disorders Amenable to Treatment

Inadequate renal production of erythropoietin can lead to hypoplastic anemia. Anemia in premature infants can develop if there is relatively low absolute reticulocyte counts or low concentration of serum erythropoietin. Renal failure or nephrectomy can lead to decreased Epo synthesis, reduced RBC numbers, and, ultimately, severe anemia as observed in predialysis and dialysis patients. Various other neoplastic disease states also are accompanied by abnormal erythropoiesis. See, e.g., Vedovato, et al., Acta.Haematol, 71, 211-213 (1984) (beta-thalassemia); Vichinsky, et al., J.Pediatr., 105(1), 15-21 (1984) (cystic fibrosis); Cotes, et al., Brit. J. Obstet. Gyneacol., 90(4), 304-311 (1983) (pregnancy, menstrual disorders); Haga, et al., Acta.Pediatr.Scand., 72, 827-831 (1983) (early anemia of prematurity); Claus-Walker, et al., Arch.Phys.Med.Rehabil., 65, 370-374 (1984) (spinal cord injury); Dunn, et al., Eur.J.Appl.Physiol., 52, 178-182 (1984) (space flight); Miller, et al., Brit. J. Haematol., 52, 545-590 (1982) (acute blood loss); Udupa, et al., J. Lab. Clin. Med., 103(4), 574-580 and 581-588 (1984); and Lipschitz, et al., Blood, 63(3), 502-509 (1983) (aging); and Dainiak, et al., Cancer, 51(6), 1101-1106 (1983) and Schwartz, et al., Otolaryngol., 109, 269-272 (1983)).

Subnormal red blood cell counts may also result from the toxic effects on erythroid precursor cells due to the use of chemotherapeutic agents or azidothymidine (AZT) in the treatment of cancers or AIDS, respectively. In addition, a variety of acquired and congenital syndromes, such as aplastic anemia, myeloproliferative syndrome, malignant lymphomas, multiple myeloma, neonatal prematurity, sickle-cell anemia, porphyria cutanea tarda, and Gaucher's disease include anemia as one clinical manifestation of the syndrome.

Recombinant human erythropoietin has been used to treat patients with a number of diseases due to abnormal Epo level or abnormal erythropoiesis, e.g., anemia due to renal failure or hemodialysis patients suffering from anemia, particularly transfusion-induced anemia. For example, Epo has been approved for the treatment of anemia associated with chemotherapy, as well as being developed for the anemia that develops as a consequence of AIDS, anemia due to prematurity and for autologous blood donation. Recombinant human Epo has also been used successfully in the treatment of advanced gastrointestinal cancer to increase the hemoglobin/hematocrit (Hgb/Hcrt) count by stimulation of red blood cell production (J. Clin. Oncology 16: 434-40, 1998). There are many reports of treatment of blood disorders characterized by low or defective red blood cell production. See, generally, Pennathur-Das, et al., Blood, 63(5), 1168-71 (1984) and Haddy, Am. J. Ped. Hematol./Oncol., 4, 191-196, (1982) relating to erythropoietin in possible therapies for sickle cell disease, and Eschbach, et al. J. Clin. Invest., 74(2), pp. 434-441, (1984), describing a therapeutic regimen for uremic sheep based on in vivo response to erythropoietin-rich plasma infusions and proposing a dosage of 10 U Epo/kg per day for 15-40 days as corrective of anemia of the type associated with chronic renal failure. See also, Krane, Henry Ford Hosp. Med. J., 31(3), 177-181 (1983).

Other than administering recombinant Epo to treat patients, there have also been methods that employ compounds that modulate Epo level in the treatment of subjects with Epo deficiency (see, e.g., U.S. Pat. No. 5,985,913). Standard methods and commercially available kits (e.g., ELISA kits) have been developed to measure level of Epo in blood. For example, Epo-ELISA kits can be obtained from R&D Systems (catalogue # DEP00, Minneapolis, Minn.).

Any of the above described diseases and disorders is suitable for treatment with the therapeutic compositions and methods of the present invention. Rather than administering exogenous Epo protein as in some of the methods described in the art, therapeutic compositions and methods of the present invention are directed to modulating (e.g., enhancing) expression of endogenous Epo gene using the novel Epo-modulators identified in accordance with the present invention.

B. Pharmaceutical Compositions

The Epo modulators of the present invention can be directly administered under sterile conditions to the subject to be treated. The modulators can be administered alone or as the active ingredient of a pharmaceutical composition. Therapeutic composition of the present invention can also be combined with or used in association with other therapeutic agents.

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, modulatory compounds or transduced cell), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the Epo modulator can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

C. Dosages and Modes of Administration

The therapeutic formulations can be delivered by any effective means that could be used for treatment. Depending on the specific Epo modulators to be administered, the suitable means include oral, rectal, vaginal, nasal, pulmonary administration, or parenteral (including subcutaneous, intramuscular, intravenous and intradermal) infusion into the bloodstream.

For parenteral administration, Epo modulators of the present invention may be formulated in a variety of ways. Aqueous solutions of the modulators may be encapsulated in polymeric beads, liposomes, nanoparticles or other injectable depot formulations known to those of skill in the art. The nucleic acids may also be encapsulated in a viral coat.

Additionally, the Epo-modulators of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The compositions may be supplemented by active pharmaceutical ingredients, where desired. Optional antibacterial, antiseptic, and antioxidant agents may also be present in the compositions where they will perform their ordinary functions.

The therapeutic formulations can conveniently be presented in unit dosage form and administered in a suitable therapeutic dose. A suitable therapeutic dose can be determined by any of the well-known methods such as clinical studies on mammalian species to determine maximum tolerable dose and on normal human subjects to determine safe dosage. Except under certain circumstances when higher dosages may be required, the preferred dosage of an Epo modulator usually lies within the range of from about 0.001 to about 1000 mg, more usually from about 0.01 to about 500 mg per day.

The preferred dosage and mode of administration of an Epo modulator can vary for different subjects, depending upon factors that can be individually reviewed by the treating physician, such as the condition or conditions to be treated, the choice of composition to be administered, including the particular Epo modulator, the age, weight, and response of the individual subject, the severity of the subject's symptoms, and the chosen route of administration. As a general rule, the quantity of an Epo modulator administered is the smallest dosage that effectively and reliably prevents or minimizes the conditions of the subjects. Therefore, the above dosage ranges are intended to provide general guidance and support for the teachings herein, but are not intended to limit the scope of the invention.

In some applications, a first Epo modulator is used in combination with a second Epo modulator in order to modulate Epo expression to a more extensive degree than cannot be achieved when one Epo modulator is used individually.

D. Delivery of Polynucleotides Encoding Epo Modulators

In some methods of the present invention, polynucleotides encoding Epo-modulating polypeptides of the present invention (e.g., those listed in Table 1, substantially identical sequences, or fragments thereof) are transfected into cells for therapeutic purposes in vitro and in vivo. In addition, novel polynucleotide modulators that can be identified in accordance with the present invention, or polynucleotides encoding novel polypeptide Epo-modulators of the invention, can also be employed in such therapeutic applications. These polynucleotides can be inserted into any of a number of well-known vectors for the transfection of target cells and organisms as described below. The nucleic acids are transfected into cells, ex vivo or in vivo, through the interaction of the vector and the target cell. The compositions are administered to a subject in an amount sufficient to elicit a therapeutic response in the subject.

Methods of modulating Epo expression by gene therapy have be described in the art. For example, U.S. Pat. No. 6,355,241 disclosed methods for in vivo production and delivery of erythropoietin. It described transfection of mammalian somatic cells with exogenous DNA encoding Epo, stable integration of the exogenous sequence into host genomes or expression of the sequence episomally, and use of the genetically engineered cells to deliver Epo to the systemic circulation of an individual in need of Epo. This resulted in an increase in mature red blood cell numbers, an increase in the oxygen-carrying potential of the blood, and an alleviation of the symptoms of anemia. Gene therapy using polynucleotides encoding the novel Epo-modulators of the present invention can be similarly carried out.

Gene therapy procedures have also been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

Delivery of the gene or genetic material into the cell is the first step in gene therapy treatment of disease. A large number of delivery methods are well known to those of skill in the art. Preferably, the polynucleotides are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In particular, a number of viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors (Ellem et al., Immunol Immunother. 44(1):10-20 (1997); Dranoffetal., Hum. Gene Ther. 1:111-2 (1997)).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc. Natl. Acad. Sci. U.S.A. 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from subjects).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

### EXAMPLES

The following examples are offered to illustrate, but not to limit the present invention.

### Example 1

### Modulation of Expression from an Epo Enhancer Element

This Example describes identification of various Epo-modulating polypeptides that up-regulate expression of a reporter gene under the control of an Epo transcription regulatory element.

The screening employed a reporter construct and a human cDNA library. The reporter construct harbors luciferase gene under the control of the 3′ enhancer of human Epo gene and a heterologous promoter HSV-1 TK (Blanchard et al., Mol. Cell. Biol. 12: 5373-85,1992; and Kung et al. 2000. Nature Medicine 6(12): 1335-40). The human cDNA collection was obtained from Incyte Genomics (Palo Alto, Calif.) and ATCC (Manassas, Va.). HepG2 cells (ATCC), at a density of 750,000 cells/ml in DMEM-3% FBS, were transiently transfected with the reporter construct and the cDNAs. Specifically, the 384 well plates used were spotted prior to transfection procedure with 62.5 ng cDNA per well and Fugene with a 6:1 Fugene to DNA ratio. 20 μl of the transient transfection mix was added to the cDNA-containing plate and incubated for 20-30 minutes for DNA complex to form. 20 μl of medium was dispensed using multi-drop and expression of the reporter gene was assayed after 48 hours. The luciferase signal (luminescence) was read by adding 40 μl 50% Bright-Glo and detecting signal immediately on an Acquest 384.1536 (LjL Biosystems Sunnyvale, Calif.) plate reader.

Results from the above screens were normalized to a mean value. The baseline is calculated as the mean of the plate. The most potent activators were identified as modulators of Epo expression. GenBank accession numbers of these modulators and the degree of up-regulation of the reporter gene expression by the modulators are shown in Table 1.

TABLE 1
Polynucleotides encoding Epo-modulating polypeptides

| Clone | | GenBank | Description of the polynucleotide | | |
|---|---|---|---|---|---|
| No. | Fold x | Acc. No. | sequence and encoded polypeptide | Notes | Domain |
| 1 | 17.6 | AF023244 | Homo sapiens osmosis | Unknown function, | TPR domains |
| | | | responsive factor mRNA, | induced by | |
| | | | complete cds | osmotic stress | |
| 2 | 17.3 | NM_021640 | Homo sapiens chromosome 12 | Melanocyte | Uncharacterized |
| | | | open reading frame 10 | proliferating gene | protein |
| | | | (C12orf10), mRNA | | family |
| 3 | 15.1 | NM_021640 | Homo sapiens chromosome 12 | Melanocyte | Uncharacterized |
| | | | open reading frame 10 | proliferating gene | protein |
| | | | (C12orf10), mRNA | | family |
| 4 | 10.8 | AF111805 | Homo sapiens MSTP024 | Melanocyte | Uncharacterized |
| | | | (MST024) mRNA, complete cds | proliferating gene | protein |
| | | | | | family |
| 5 | 9.5 | BC016957 | Homo sapiens, synovial | | Krab box |
| | | | sarcoma, X breakpoint 2, clone | | |
| | | | MGC: 21532 IMAGE: 3916128, | | |
| | | | mRNA, complete cds | | |
| 6 | 6.4 | AJ006422 | HSA6422Homo sapiens mRNA | | ArfGap and |
| | | | for centaurin-alpha | | PH |
| 7 | 5.6 | BC009093 | Mus musculus, early growth | Egr2 | Zn finger |
| | | | response 2, clone MGC: 7113 | | |
| | | | IMAGE: 3157863, mRNA, | | |
| | | | complete cds | | |
| 8 | 5.6 | BC001485 | Homo sapiens, ras-related C3 | | Rho, Ras |
| | | | botulinum toxin substrate 2 (rho | | domain |
| | | | family, small GTP binding protein | | |
| | | | Rac2), clone MGC: 1383 | | |
| | | | IMAGE: 3509104, mRNA, | | |
| | | | complete cds | | |
| 9 | 5.6 | BC002613 | Homo sapiens, clone | | |
| | | | IMAGE: 3161564, mRNA, partial | | |
| | | | cds | | |
| 10 | 5.5 | BC005695 | Mus musculus, Similar to | Iron binding | Ard |
| | | | hypothetical protein FLJ10913, | domain | |
| | | | clone MGC: 11855 | | |
| | | | IMAGE: 3597652, mRNA, | | |
| | | | complete cds | | |
| 11 | 5.5 | AB014516 | Homo sapiens mRNA for | Also hits in CREB | |
| | | | KIAA0616 protein, partial cds | screen | |
| 12 | 5.0 | NM_004430 | Homo sapiens early growth | Egr3 | Zn finger |
| | | | response 3 (EGR3), mRNA | | |
| 13 | 4.9 | BC020028 | Mus musculus, Similar to | | Mob1_phocein |
| | | | hypothetical protein FLJ13204, | | |
| | | | clone MGC: 28259 | | |
| | | | IMAGE: 4007373, mRNA, | | |
| | | | complete cds | | |
| 14 | 4.9 | BC012870 | Mus musculus, Similar to aryl | | bHLH-PAS |
| | | | hydrocarbon receptor nuclear | | domains |
| | | | translocator, clone MGC: 11512 | | |
| | | | IMAGE: 3964932, mRNA, | | |
| | | | complete cds | | |
| 15 | 4.9 | AF023244 | Homo sapiens osmosis | Unknown function, | TPR domains |
| | | | responsive factor mRNA, | induced by | |
| | | | complete cds | osmotic stress | |
| 16 | 4.8 | AP003025 | Homo sapiens genomic DNA, | | |
| | | | chromosome 11q, clone: RP11217L21, | | |
| | | | complete sequence | | |
| 17 | 4.8 | BC005455 | Mus musculus, RAS-related C3 | | Rho, Ras |
| | | | botulinum substrate 2, clone | | domain |
| | | | MGC: 6255 IMAGE: 3495818, | | |
| | | | mRNA, complete cds | | |
| 18 | 4.5 | BC002150 | Mus musculus, Similar to single- | | SSDP |
| | | | stranded-DNA-binding protein, | | |
| | | | clone MGC: 7269 | | |
| | | | IMAGE: 3484896, mRNA, | | |
| | | | complete cds | | |
| 19 | 4.5 | NM_002032 | Homo sapiens ferritin, heavy | Iron binding | ferritin |
| | | | polypeptide 1 (FTH1), mRNA | | |
| 20 | 4.3 | BC020988 | Homo sapiens, clone MGC: 9730 | Hypothetical | exonuclease |
| | | | IMAGE: 3852419, mRNA, | protein FLJ12484 | |
| | | | complete cds | | |
| 21 | 4.2 | M31222 | HUME12A Human e12 protein | HLH transcription | HLH |
| | | | (E2A) mRNA, complete cds | factor | |
| 22 | 4.1 | BC013435 | Homo sapiens, Similar to | Hypothetical | |
| | | | hypothetical protein FLJ11467, | protein FLJ11467 | |
| | | | clone MGC: 16891 | | |
| | | | IMAGE: 3892997, mRNA, | | |
| | | | complete cds | | |
| 23 | 4.1 | NM_005957 | Homo sapiens partial MTHFR | NADPH reductase | MTHFR |
| | | | gene for | | |
| | | | methylenetetrahydrofolate | | |
| 24 | 4.0 | BC015506 | Homo sapiens, peroxisomal | membrane vesicle | Peroxisomal |
| | | | biogenesis factor 3, clone | assembly | biogenesis |
| | | | MGC: 9125 IMAGE: 3914956, | | factor 3 |
| | | | mRNA, complete cds | | (Peroxin-3) |
| 25 | 4.0 | BC011482 | Mus musculus, Similar to | membrane vesicle | C2 |
| | | | membrane bound C2 domain | assembly | |
| | | | containing protein, clone | | |
| | | | MGC: 18775 IMAGE: 4165946, | | |
| | | | mRNA, complete cds | | |
| 26 | 4.0 | Z82254 | Homo sapiens similar to RIKEN | similar to | |
| | | | cDNA 2810028A01 | paraneoplastic | |
| | | | (LOC257051), mRNA | antigen MA1 | |
| 27 | 3.9 | Z82254 | Homo sapiens similar to RIKEN | similar to | |
| | | | cDNA 2810028A01 | paraneoplastic | |
| | | | (LOC257051), mRNA | antigen MA1 | |
| 28 | 3.9 | BC001214 | Homo sapiens, CGI-68 protein, | Protein-leucine O- | LCM |
| | | | clone MGC: 682 | methyltransferase | |
| | | | IMAGE: 3533656, mRNA, | | |
| | | | complete cds | | |
| 29 | 3.9 | BC003316 | Mus musculus, TGFB inducible | Egr1 | Zn finger |
| | | | early growth response, clone | | |
| | | | MGC: 7000 IMAGE: 3155247, | | |
| | | | mRNA, complete cds | | |
| 30 | 3.8 | BC019981 | Mus musculus, Similar to | nucleolar RNA | |
| | | | hypothetical protein FLJ21959, | associated protein | |
| | | | clone MGC: 28409 | | |
| | | | IMAGE: 4035854, mRNA, | | |
| | | | complete cds | | |
| 31 | 3.8 | BC003361 | Homo sapiens, solute carrier | solute carrier | Pdz |
| | | | family 9 (sodium/hydrogen | regulatory factor | |
| | | | exchanger), isoform 3 regulatory | | |
| | | | factor 1, clone MGC: 5270 | | |
| | | | IMAGE: 2900516, mRNA, | | |
| | | | complete cds | | |
| 32 | 3.8 | BC010496 | Mus musculus, Tial1 cytotoxic | RNA binding | RRM |
| | | | granule-associated RNA binding | protein | |
| | | | protein-like 1, clone MGC: 5930 | | |
| | | | IMAGE: 3497971, mRNA, | | |
| | | | complete cds | | |
| 33 | 3.5 | BC003855 | Mus musculus, clone MGC: 6439 | MMTV | MMTV |
| | | | IMAGE: 3601769, mRNA, | superantigen | superantigen |
| | | | complete cds | | |
| 34 | 3.5 | BC004603 | Mus musculus, Similar to RIKEN | | |
| | | | cDNA 2010003O02 gene, clone | | |
| | | | MGC: 7975 IMAGE: 3585190, | | |
| | | | mRNA, complete cds | | |
| 35 | 3.5 | BC006746 | Mus musculus, Similar to | catalyzes the last | Ferrochelatase |
| | | | ferrochelatase, clone MGC: 6205 | step in heme | |
| | | | IMAGE: 3256604, mRNA, | biosynthesis | |
| | | | complete cds | | |
| 36 | 3.4 | BC006642 | Mus musculus, nasal embryonic | negative | |
| | | | LHRH factor, clone MGC: 7230 | elongation factor | |
| | | | IMAGE: 3483536, mRNA, | | |
| | | | complete cds | | |
| 37 | 3.4 | BC016283 | Homo sapiens, clone MGC: 9023 | iron binding | ABC |
| | | | IMAGE: 3909151, mRNA, | | transporter, |
| | | | complete cds | | iron binding |
| 38 | 3.4 | AF429308 | Homo sapiens testis specific | sulfurtransferase | Rhodanese |
| | | | protein A14 I-type (TSGA14) | involved in | domain |
| | | | mRNA, complete cds; | cyanide | |
| | | | alternatively spliced | detoxification | |
| 39 | 3.4 | NM_002774 | Homo sapiens kallikrein 6 | protease | Trypsin |
| | | | (neurosin, zyme) (KLK6), mRNA | | |
| 40 | 3.2 | AK055331 | Homo sapiens cDNA FLJ30769 | Cab45, calcium | EF hand |
| | | | fis, clone FEBRA2000733, highly | binding protein | |
| | | | similar to 45 KDA CALCIUM- | | |
| | | | BINDING PROTEIN | | |
| | | | PRECURSOR | | |
| 41 | 3.2 | NM_024698 | Homo sapiens solute carrier | substrate carrier | mito_carr |
| | | | family 25 (mitochondrial carrier: | proteins that are | |
| | | | glutamate), member 22 | involved in energy | |
| | | | (SLC25A22), mRNA | transfer | |
| 42 | 3.2 | BC015857 | Homo sapiens, Similar to RIKEN | | |
| | | | cDNA 5730589L02 gene, clone | | |
| | | | IMAGE: 4671596, mRNA | | |
| 43 | 2.9 | NM_002032 | Homo sapiens ferritin, heavy | Iron binding | ferritin |
| | | | polypeptide 1 (FTH1), mRNA | | |
| 44 | 2.8 | BC002727 | Homo sapiens, hypothetical | Syntenin 2, may | Pdz |
| | | | protein, clone MGC: 3734 | target signaling | |
| | | | IMAGE: 3633262, mRNA, | proteins to | |
| | | | complete cds | membranes | |
| 45 | 2.8 | AF131809 | Homo sapiens clone 24904 | Syntenin 2, may | Pdz |
| | | | mRNA sequence, complete cds | target signaling | |
| | | | | proteins to | |
| | | | | membranes | |
| 46 | 2.7 | AL121749 | Human DNA sequence from | frizzled 8, | Fz domain, |
| | | | clone RP11-425A6 on | | Frizzled |
| | | | chromosome 10, complete | | smoothened |
| | | | sequence | | family |
| | | | | | membrane |
| | | | | | region |
| 47 | 2.6 | BC003162 | Homo sapiens, lamin A/C, clone | lamin, chromatin | Intermediate |
| | | | MGC: 4219 IMAGE: 2958052, | structure, nuclear | filament |
| | | | mRNA, complete cds | membrane | protein, |
| | | | | | intermediate |
| | | | | | filament tail |
| | | | | | domain |
| 48 | 2.5 | NM_019101 | Homo sapiens apolipoprotein M | | |
| | | | (APOM), mRNA | | |
| 49 | 2.5 | AK057586 | Homo sapiens cDNA FLJ33024 | | |
| | | | fis, clone THYMU1000532, | | |
| | | | moderately similar to HIGH- | | |
| | | | AFFINITY CAMP-SPECIFIC | | |
| | | | 3′,5′-CYCLIC | | |
| | | | PHOSPHODIESTERASE (EC | | |
| | | | 3.1.4.17) | | |

### Example 2

### Characterization of Epo-modulating Polypeptides in Hypoxia

This Example describes activation of the Epo enhancer by several Epo-modulating polypeptides via stabilization of Hif1-α.

Hypoxia is a condition of low oxygen tension that has a physiological role in many disease states and biological functions such as erythropoiesis and angiogenesis. In cells, a response to this condition is regulated via a DNA motif found in targeted genes. Epo is one such gene and the 3′ element is known as the Hypoxia Response Element (HRE). This element is bound by the Hif1 complex which activates downstream genes. Hif1 is a multimeric complex composed of HIF1-α and ARNT. Under normoxic conditions, HIF1-α is targeted for degradation via a proteasome/ubiquitination pathway. By screening hundreds of cDNAs against an Epo reporter containing the HRE, a better understanding of proteins which interact with HIF1-α can be achieved. These select proteins were then analyzed for their ability to stabilize the HIF1-α protein.

After the initial screening as described in Example 1, the top hits were subject to a secondary screening. HepG2 cells at a density of 500,000 cells/well were transiently transfected with 3.5 μg of the appropriate cDNA using Fugene6 at a ratio of 6:1. Cells were harvested 48 hr post transfection in ice-cold phosphate buffered saline (PBS). Cells were spun 3,300 rpm for 2 minutes and resuspended in PBS containing a protease inhibitor cocktail. The samples were sonicated, then loaded on a 4-12% Bis-Tris protein gel. Analyses via western blot were performed using standard procedures.

From the cDNA collections, a total of 49 clones were chosen for further analysis. These clones showed activity against the Epo reporter between 2.5-17 fold over control-transfected cells in a secondary screen. Each clone was then sequenced to confirm its identity. The final filter for specificity required that the selected cDNA had the ability to stabilize Hif1-α under normoxic condtions when transiently transfected into HepG2 cells. Among this set of 49 clones, several clones that scored highest in their ability to stabilize Hif1-α are all molecules shown in Table 1. These include polynucleotides with accession numbers AL121749, AF429308, BC001214, BC015506, BC01482, BC013435, BC020988, and NM 021640.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

1. A method for identifying an agent that modulates expression of erythropoietin (Epo), the method comprising:
(a) assaying a biological activity of an Epo-modulating polypeptide encoded by a polynucleotide selected from the members listed in Table 1, or a fragment of said polypeptide, in the presence of a test agent to identify one or more modulating agents that modulate the biological activity of the polypeptide; and
(b) testing one or more of the modulating agents for ability to modulate expression of Epo; thereby identifying an agent that modulates expression of Epo.

2. The method of claim 1, wherein (b) comprises testing the modulating agents for ability to modulate expression of a reporter gene under the control of an Epo transcription regulatory element.

3. The method of claim 2, wherein the Epo transcription regulatory element is a 3′ enhancer element.

4. The method of claim 3, wherein the 3′ enhancer element is a human Epo enhancer.

5. The method of claim 1, wherein the Epo-modulating polypeptide is encoded by a polynucleotide having an accession number that is selected from the group consisting of AL121749, AF429308, BC001214, BC015506, BC011482, BC013435, BC020988, and NM 021640.

6. The method of claim 1, wherein the Epo-modulating polypeptide is a transcription regulator and the biological activity is transcriptional regulation.

7. The method of claim 1, wherein the Epo-modulating polypeptide is an enzyme and the biological activity is an enzymatic activity of the Epo-modulating polypeptide.

8. The method of claim 1, wherein the test agent modulates cellular level of the Epo-modulating polypeptide.

9. The method of claim 1, wherein the modulating agent identified in (b) enhances Epo expression.

10. The method of claim 1, wherein the assaying of the biological activity of the Epo-modulating polypeptide occurs in a cell.

11. The method of claim 10, wherein the Epo-modulating polypeptide is expressed from said polynucleotide that has been introduced into the cell.

12. The method of claim 1, wherein the testing for ability to modulate Epo expression comprises:
providing a cell or cell lysate that comprises a reporter gene that is operably linked to an Epo transcription regulatory element;
contacting the cell or cell lysate with a modulating agent; and
detecting an increase or decrease in expression of the reporter gene in the presence of the modulating agent compared to expression of the reporter gene in the absence of the modulating agent.

13. The method of claim 1, wherein the testing for ability to modulate expression of Epo comprises contacting a cell or cell lysate with the modulating agent and determining cellular level of Epo or a fragment of Epo.

14. A method for modulating Epo expression in a cell, comprising contacting the cell with (a) an Epo-modulating polypeptide encoded by a polynucleotide selected from the members listed in Table 1, or a fragment of the Epo-modulating polypeptide, or (b) an Epo modulator identified in claim 1; thereby modulating Epo expression in the cell.

15. The method of claim 14, wherein the Epo is human Epo.

16. The method of claim 14, wherein the cell is a kidney cell or a liver cell.

17. The method of claim 14, wherein the cell is present in a subject.

18. The method of claim 17, wherein the subject is human.

19. The method of claim 14, wherein the Epo-modulating polypeptide or its fragment is expressed from an expression vector that has been introduced into the cell.

20. The method of claim 14, wherein the Epo modulator identified in claim 1 enhances Epo expression.

21. The method of claim 14, wherein the Epo modulator is a small molecule compound.

22. A method for treating erythropoietin deficiency in a subject, the methods comprising administering to the subject a pharmaceutical composition comprising an effective amount of an Epo-modulator that enhances Epo expression, thereby treating Epo deficiency in the subject;
wherein the Epo-modulator is identified by (a) assaying a biological activity of an Epo-modulating polypeptide encoded by a polynucleotide selected from the members listed in Table 1, or a fragment of said polypeptide, in the presence of a test agent to identify one or more modulating agents that modulate the biological activity of the polypeptide; and
(b) testing one or more of the modulating agents for ability to enhance expression of Epo gene.

23. The method of claim 22, wherein the subject is a human, and the Epo is human Epo.

24. The method of claim 22, wherein the Epo modulator is a small molecule compound.

25. The method of claim 22, wherein the Epo-modulating polypeptide is a transcription regulator.

26. The method of claim 22, wherein the Epo-modulating polypeptide is an enzyme.

27. The method of claim 22, wherein the subject suffers from anemia.
